(19) United States
(12) Patent Application Publication
(10) Pub. No.: US 2026/0306069 A1
(43) Pub. Date: Oct. 8, 2026

(54) AUTOMATIC STEERING FOR ULTRASOUND IMAGING USING ARTIFICIAL INTELLIGENCE

(71) Applicant: GE Precision Healthcare LLC, Waukesha, WI (US)

(72) Inventor: Dagfinn Saetre, Tønsberg, Vestfold (NO)

(73) Assignee: GE Precision Healthcare LLC, Waukesha, WI (US)

(21) Appl. No.: 19/098,759

(22) Filed: Apr. 2, 2025

(51) Int. Cl. A61B 8/00

(52) U.S. Cl. CPC A61B 8/54; A61B 8/42; A61B 8/461; A61B 8/5207

(57) ABSTRACT

An ultrasound imaging system includes a transducer configured to transmit and receive an ultrasound signal in a three-dimensional volume. The ultrasound imaging system includes a processing circuit having a processor coupled to a memory device storing instructions thereon that, when executed, cause the processing circuit to perform operations including receiving first image data obtained by an ultrasound probe from the three-dimensional volume, the first image data corresponding with a first plane in the volume, identifying a desired view based on the first image data, determining, by an artificial intelligence model, a deviation from the first plane to the desired view based on the first image data, automatically applying the deviation to the ultrasound signal to receive second image data corresponding with a second plane based on the desired view, and displaying an output image based on the second image data on a display screen of the ultrasound imaging system.

### FIELD

Embodiments of the subject matter disclosed herein relate to ultrasound imaging, and more particularly, to the use of artificial intelligence and ultrasound imaging to automatically steer an ultrasound signal.

### BACKGROUND

During a medical imaging scan, a plurality of medical images of a patient are obtained by a technician, such as a sonographer, to measure or detect various aspects of anatomical features present within the medical images. Obtaining medical images of a standard view is critical for identifying anatomical features and diagnosis of the patient. Obtaining the standard view may be challenging for the technician and require manual adjustment of the probe until the standard view is obtained.

### SUMMARY

An embodiment relates to an ultrasound imaging system. The ultrasound imaging system includes a transducer configured to transmit and receive an ultrasound signal in a three-dimensional volume. The three-dimensional volume includes a plurality of planes. The ultrasound imaging system includes a processing circuit. The processing circuit includes a processor coupled to a memory device storing instructions thereon that, when executed, cause the processing circuit to perform operations including receiving first image data obtained by an ultrasound probe from the three-dimensional volume, the first image data corresponding with a first plane in the volume, identifying a desired view based on the first image data, determining, by an artificial intelligence model, a deviation from the first plane to the desired view based on the first image data, automatically applying the deviation to the ultrasound signal to receive second image data corresponding with a second plane based on the desired view, and displaying an output image based on the second image data on a display screen of the ultrasound imaging system.

Another embodiment relates to a medical imaging system. The medical imaging system includes an imaging unit configured to transmit and receive an ultrasound signal in a three-dimensional volume to obtain first image data along a first plane. The medical imaging system includes a control circuit configured to determine, by an artificial intelligence model, a deviation from the first plane to a desired view based on the first image data. The deviation is automatically applied to the ultrasound signal to receive second image data corresponding with a second plane based on the desired view, such that the medical imaging system is configured to display an output image based on the second image data on a display screen of the medical imaging system.

Another embodiment relates to a method. The method includes receiving, by a processing circuit, first image data obtained by an ultrasound probe from a three-dimensional volume. The first image data corresponds with a first plane in the volume. The method includes identifying, by the processing circuit, a desired view based on the first image data. The method includes determining, by the processing circuit and an artificial intelligence model, a deviation from the first plane to the desired view based on the first image data. The method includes automatically applying, by the processing circuit, the deviation to an ultrasound signal to receive second image data corresponding with a second plane along the desired view. The method displaying an output image based on the second image data on a display screen of an ultrasound imaging system.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE DRAWINGS

FIG. 1 is a block diagram of an ultrasound imaging system, according to an example embodiment.

FIG. 2 is a block diagram of a flow diagram using an artificial intelligence (AI) circuit of the ultrasound imaging system of FIG. 1, according to an example embodiment.

FIG. 3 is an illustration of an ultrasound imaging probe, according to an example embodiment.

FIG. 4 is another illustration of an ultrasound imaging probe, according to an example embodiment.

FIG. 5 is an illustration of a scan volume of the ultrasound imaging probe of FIG. 4, according to an example embodiment.

FIG. 6 is a flow chart illustrating a method for ultrasound imaging using the ultrasound imaging system of FIG. 1, according to an example embodiment.

FIG. 7 is a flow chart illustrating a method for using an artificial intelligence model, according to an example embodiment.

FIG. 8A is an illustration of an acquired ultrasound image, according to an example embodiment.

FIG. 8B is another illustration of an acquired ultrasound image, according to an example embodiment.

FIG. 8C is another illustration of an acquired ultrasound image, according to an example embodiment.

### DETAILED DESCRIPTION

Referring generally to the figures, systems and methods for the use of artificial intelligence and three-dimensional ultrasound imaging to automatically steer an ultrasound signal are disclosed. The systems disclosed herein are used to receive image data corresponding with a first plane and determine a deviation from the first plane to a desired view with an artificial intelligence model. The systems and methods automatically apply the deviation to the ultrasound signal to receive second image data corresponding with a second plane based on the desired view. An output image based on the second image data can be displayed on a display screen.

During an ultrasound scan, an ultrasound probe is conventionally positioned in a sagittal orientation (e.g., pointed towards a patient's head). Generally, however, obtaining a standard view for an ultrasound scan is challenging and an acquired image at a first orientation may not be of sufficient quality to adequately view anatomical features and a state of a patient, such that the probe must be adjusted to more closely align with the standard view. Visualization of an ultrasound image in a proper standard view may be critical to make a proper diagnosis, evaluate anatomical features, and search for pathologies. For example, for a cardiac ultrasound, obtaining a standard (e.g., correctly aligned) Apical 4 chamber view of a heart of the patient is critical for obtaining accurate measurements and to make a correct diagnosis. Thus, it is desired to adjust the probe such that resultant image data from an ultrasound beam from the probe is in the proper standard view. However, manually adjusting the ultrasound probe without assistance such that the resultant image data is in the standard view is time consuming and relies on the skills and expertise of the operator (e.g., sonographer, technician, clinician, etc.), thereby resulting in inconsistencies due to operator efficiencies and in obtaining consistent and high-quality ultrasound images.

The systems and methods described herein provide a technical solution to existing ultrasound imaging systems and employ a three-dimensional ultrasound probe. The use of the three-dimensional ultrasound probe allows for a desired view (e.g., the standard view) to be determined. Additionally, the use of the three-dimensional probe allows for a deviation from current image data to the desired view to be determined. Additionally, as described herein, a scan volume from three-dimensional imaging allows for a variety of electronic manipulation techniques to be applied to adjust the image data. The scan volume may be acquired from the three-dimensional probe and the desired view can be searched within the three-dimensional volume.

Additionally, ultrasound systems may utilize guidance tools powered by artificial intelligence (AI) that aid in determining the desired view and/or determining the deviation. AI guidance tools may require or utilize less data relative to a human to analyze a current ultrasound image and generate outputs that allow for adjustment of the ultrasound image. With such AI guidance tools, the user is relieved of the responsibility of navigating the probe without aid. Further, when the AI systems are utilized, a maximum image quality may be used or required only when recording a sequence of ultrasound images for diagnostic purposes. Consequently, having fixed acquisition parameters may consume unnecessarily high amounts of an energy-transmission budget and wastes the limited battery capacity of wireless probes. Technically and beneficially, the systems and methods described herein provide faster and accurate ultrasound images, which improve productivity and diagnostic capabilities.

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

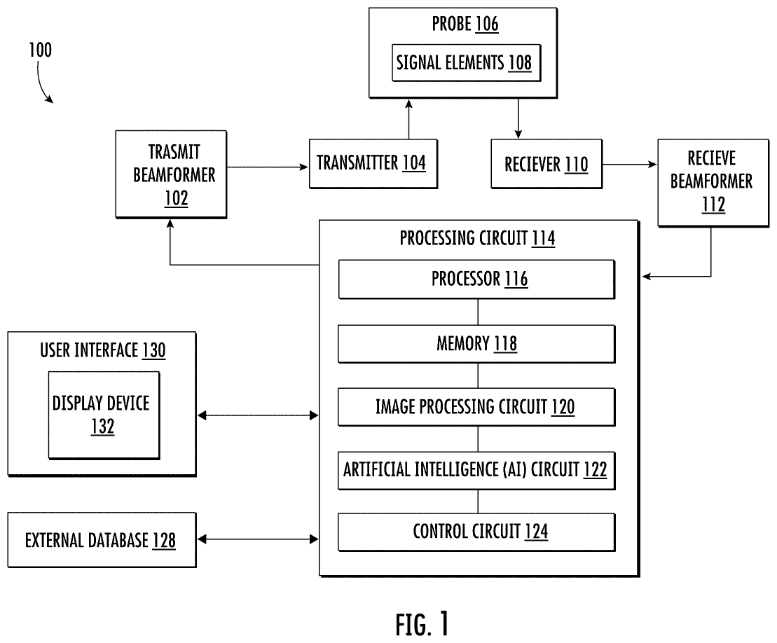

Referring to FIG. 1, a schematic diagram of an ultrasound imaging system 100 is shown. The ultrasound imaging system 100 may be used in a medical environment (e.g., hospitals, clinics, etc.), for example, by a sonographer, technician, or other clinician certified to collect ultrasound data from a patient. Although the systems and methods are described herein in the context of the ultrasound imaging system 100, it should be appreciated that the medical imaging parameter recommendation tool described herein may be implemented using any of a variety of medical imaging systems (e.g., medical resonance imaging, x-ray, computed tomography, positron emission tomography, etc.).

An example of a procedure performed using the ultrasound imaging system 100 may be an echocardiogram. Echocardiograms are performed to detect heart abnormalities in a patient by collecting and processing ultrasound data (e.g., using the ultrasound imaging system 100, as described herein). During an echocardiogram, a sonographer follows a particular imaging protocol specific to echocardiography. The echocardiography-specific imaging protocol ensures that the heart is thoroughly captured by the ultrasound data and that the processing of the ultrasound data is focused on detecting heart abnormalities. The sonographer collects the ultrasound data by navigating a probe (e.g., probe 106, as described below) over the patient's chest until a sufficient volume of ultrasound images are collected. With three-dimensional imaging, using the ultrasound imaging system 100, a three-dimensional volume of ultrasound images may be obtained. The sonographer may collect the ultrasound images both with the ultrasound probe in a sagittal orientation, a transverse orientation, and a coronal orientation. With reference to the Cartesian coordinate system described herein, the transverse orientation refers to the XZ plane or the YZ plane. The sagittal orientation refers to the other of the XZ plane or the YZ plane. The coronal orientation refers to the XY plane. The collected images are stored in a central storage device (e.g., memory 118) and analyzed by the sonographer. The sonographer generates a set of measurements from the images (e.g., 50-100 records), and the images and measurements are collectively reviewed by a cardiologist. The cardiologist provides any clinical findings/conclusions in a report submitted to the patient's medical record.

As shown in FIG. 1, the ultrasound imaging system 100 includes a transmit beamformer 102, a transmitter 104, a probe 106, a receiver 110, and a receive beamformer 112. The ultrasound imaging system 100 may also include a matching layer and a damping block. The matching layer improves ultrasound image quality by having an acoustic impedance between a tissue to be imaged and a material of a transducer of the ultrasound imaging system 100. The damping block is configured to absorb ultrasound energy to improve ultrasound image quality.

The transmit beamformer 102 may be either a hardware beamformer or a software beamformer. In embodiments where the transmit beamformer 102 is a hardware beamformer, the transmit beamformer 102 may include one or more of a graphics processing unit (GPU), a microprocessor, a central processing unit (CPU), a digital signal processor (DSP), or any other type of processor capable of performing logical operations. The transmit beamformer 102 may be configured to perform conventional beamforming techniques as well as techniques such as retrospective transmit beamforming (RTB). Alternatively, in embodiments where the transmit beamformer 102 is a software beamformer, a processor (e.g., processor 116, as described below) may be configured to perform some or all of the functions associated with the transmit beamformer 102.

The probe 106 may be a linear array probe, a curvilinear array probe, a sector probe, or any other type of probe configured to obtain two-dimensional (2D) B-mode data, 2D color flow data, M-mode data, three-dimensional (3D) data, four-dimensional (4D) data, or any other type of ultrasound data. Alternatively or additionally, the probe 106 may be any type of probe including a matrix transducer array configured to obtain three-dimensional (3D) ultrasound data. In some embodiments, the probe 106 may include a position sensor configured to detect a position of the probe 106 relative to one or more reference locations. That is, the position sensor may continuously track movement (e.g., rotation, translation, orientation, etc.) of the probe 106 relative to the location of the probe 106 when the anatomy being imaged is identified. For example, the anatomy being imaged may be identified as a left atrial appendage (LAA) at a first location of the probe 106. Then, the position sensor may track the movement of the probe 106 relative to the LAA in order to identify successive locations of the probe 106. In some embodiments, the position sensor may transmit position data to be stored within the ultrasound imaging system 100 (e.g., in memory 118).

The probe 106 may include a transducer configured to transmit and receive an ultrasound signal. In some embodiments, as shown in FIG. 1, the probe 106 includes signal elements 108.

The signal elements 108 may be arranged in a transducer array, and in some embodiments may be arranged in a 2D array. As described herein, the 2D array of the signal elements 108 may allow for 3D ultrasound imaging (e.g., such that the transducer is configured to transmit and receive a 3D ultrasound signal). The transmit beamformer 102 and the transmitter 104 drive the signal elements 108 to emit pulsed ultrasonic signals into a body of a subject (e.g., a patient). For example, during an echocardiogram, a sonographer or other clinician may navigate the probe 106 over a patient's chest so that the signal elements 108 in the probe 106 emit the pulsed ultrasonic signals into the patient's thoracic cavity. The pulsed ultrasonic signals are then back-scattered from anatomical structures in the body, such as blood cells or muscular tissues, to produce echoes that return to the signal elements 108. That is, the signal elements 108 may include the transducer configured to transmit and receive the ultrasound signal, a matching layer configured to have an acoustic impedance between a tissue to be imaged and a material of the transducer (e.g., such that the pulsed electronic signals can be back-scattered from the anatomical structures in the body and received as echoes by the signal elements 108), and a damping block configured to absorb ultrasound energy.

Additionally, in ultrasound imaging systems with electronically steered beams, the transmit beamformer 102 may be adjusted to shift an angle of the ultrasound beam, therefore shifting the ultrasound signal transmitted. For example, the transmit beamformer 102 may adjust timing and phase shifts of the ultrasound signals sent into the body of the subject to change the angle of the ultrasound signal. In various embodiments, the adjustment may occur without the operator having to move the transducer.

The receiver 110 receives the echoes from the probe 106 and converts the echoes into electrical signals. The electrical signals are then passed through the receive beamformer 112, which produces the ultrasound data from the electrical signals. As described above with reference to the transmit beamformer 102, the receive beamformer 112 may be either a hardware beamformer or a software beamformer. In embodiments where the receive beamformer 112 is a hardware beamformer, the receive beamformer 112 may include one or more of a GPU, a microprocessor, a CPU, a DSP, or any other type of processor capable of performing logical operations. The receive beamformer 112 may be configured to perform conventional beamforming techniques as well as techniques such as retrospective transmit beamforming (RTB). Alternatively, in embodiments where the receive beamformer 112 is a software beamformer, a processor (e.g., processor 116, as described below) may be configured to perform some or all of the functions associated with the receive beamformer 112.

Although the transmit beamformer 102, the transmitter 104, the receiver 110, and the receive beamformer 112 are shown in FIG. 1 as being components of the ultrasound imaging system 100 that are distinct from the probe 106, it should be appreciated that in some embodiments, the probe 106 may include electronic circuitry configured to perform the functions of each of the transmit beamformer 102, the transmitter 104, the receiver 110, and/or the receive beamformer 112. That is, all or part of the transmit beamformer 102, the transmitter 104, the receiver 110, and/or the receive beamformer 112 may be situated within the probe 106.

Referring still to FIG. 1, the ultrasound imaging system 100 is shown to include a processing circuit 114. As shown, the processing circuit 114 may include at least one processor 116, a memory 118, an image processing circuit 120, an artificial intelligence (AI) circuit 122, and a control circuit 124. In this way, the processing circuit 114 may be structured or configured to execute or implement the instructions, commands, and/or control processes described herein with respect to the processor 116, the memory 118, the image processing circuit 120, the AI circuit 122, and the control circuit 124. While shown as being separate from the probe 106 in FIG. 1, it will be appreciated that the processing circuit 114 can be part of the probe 106. For example, the processing circuit 114 can be disposed in a handheld housing of the probe 106 (e.g., in the case of the probe 106 being a wireless probe).

The processor 116 may include a CPU, a GPU, a microprocessor, a DSP, a general-purpose single-or multi-chip processor, a field-programmable gate array (FPGA), or any other type of processor capable of performing logical operations. A general-purpose processor may be a microprocessor, or, any conventional processor, or state machine. A processor also may be implemented as a combination of computing devices, such as a combination of a DSP and a microprocessor, a plurality of microprocessors, one or more microprocessors in conjunction with a DSP core, or any other such configuration. In some embodiments, the processor 116 may be shared by multiple circuits (e.g., the circuits of the processor 116 may include or otherwise share the same processor which, in some example embodiments, may execute instructions stored, or otherwise accessed, via different areas of the memory 118). Alternatively or additionally, the processor 116 may be structured to perform or otherwise execute certain operations independent of one or more co-processors. In some embodiments, two or more processors may be coupled via a bus to enable independent, parallel, pipelined, or multi-threaded instruction execution. All such variations are intended to fall within the scope of the present disclosure.

The processor 116 may be configured to control the transmit beamformer 102, the transmitter 104, the receiver 110, and the receive beamformer 112. The processor 116 may also be in electronic communication with the probe 106. For purposes of this disclosure, the term “electronic communication” may be defined to include both wired and wireless communications.

In some embodiments, the processor 116 may be configured to control the probe 106 during data acquisition. That is, the processor 116 may control the data acquisition by controlling which of the signal elements 108 are active and by controlling a shape of the beam emitted from the probe 106. For example, using a matrix configuration of the signal elements 108, the processor 116 may be configured to control a transmit direction of the probe 106 such that data is acquired from a sagittal plane and/or a transverse plane of the probe 106. In another example, for 3D ultrasound imaging, the processor 116 may be configured to automatically steer the ultrasound signal to acquire data along a plane within a volume. According to other embodiments, the transmit direction of the probe 106 may be controlled by another component of the ultrasound imaging system 100 (e.g., the control circuit 124).

Alternatively or additionally, the processor 116 may include a complex demodulator configured to demodulate radio frequency (RF) data obtained by the probe 106 and generate raw data. According to other embodiments, the demodulation of the RF data may be performed by another component of the ultrasound imaging system 100. The processor 116 may perform the processing operations described herein according to a plurality of selectable ultrasound modalities.

Depending on a mode of operation of the ultrasound imaging system 100, the processor 116 may process ultrasound data obtained by the probe 106 according to the mode of operation to generate image data. For example, the mode of operation may include B-mode, color flow Doppler mode, M-mode, color M-mode, spectral Doppler, elastography, TVI, strain, strain rate, and the like. Various of these modes of operation may be configured to, for instance, convert ultrasound data from beam space coordinates (e.g., received from the receive beamformer 112) to display space coordinates (e.g., such that the ultrasound data may be displayed as image data). In some embodiments, the mode of operation may allow for video processing by the processor 116 such that a series of images (e.g., processed ultrasound data) may be displayed in real-time while a scanning session/procedure is being performed on a patient. An operator of the ultrasound imaging system 100 (e.g., a sonographer) may switch between various modes in order to obtain a variety of ultrasound data and to perform a complete scan of an anatomical region of interest. For example, the operator may switch between modes using user interface 130 (e.g., using physical controls, interface inputs representing physical controls, etc.).

The processor 116 performs the processing operations in real-time as the echo signals are received by the receiver 110 from the probe 106. For the purposes of this disclosure, the term “real-time” is defined to include a procedure that is performed without any intentional delay. As an illustrative, non-limiting example, in certain instances, the ultrasound imaging system 100 may obtain images at a real-time volume-rate of 7-20 volumes/sec. It should be appreciated, however, that the real-time volume-rate may be dependent on the length of time that it takes to obtain each volume of data for display. Thus, the ultrasound imaging system 100 may be configured to obtain 2D data of an anatomical region at a faster rate than 3D data of the same anatomical region because it takes longer to obtain a volume of 3D data than the same volume of 2D data. Similarly, when the ultrasound imaging system 100 obtains a relatively large volume of data, the real-time volume-rate may be slower than for a smaller volume of data. For example, during an abdominal scan, the real-time volume-rate may be slower if the patient is an adult versus if the patient is an infant because the volume of data is larger for the adult than for the infant (e.g., due to the abdomen of an adult being larger than the abdomen of an infant). Therefore, certain implementations of the ultrasound imaging system 100 may have real-time volume-rates that are faster than 20 volumes/sec, while other implementations of the ultrasound imaging system 100 may have real-time volume-rates that are slower than 7 volumes/sec.

In some embodiments, the ultrasound imaging system 100 may include multiple processors configured to perform the processing operations/functionality described with reference to processor 116. For example, in such embodiments, a first processor of the multiple processors may be configured to demodulate and decimate the RF signal while a second processor of the multiple processors may be configured to further process the RF data prior to displaying an image representative of the data. It should be appreciated that other embodiments may use a different arrangement of processors.

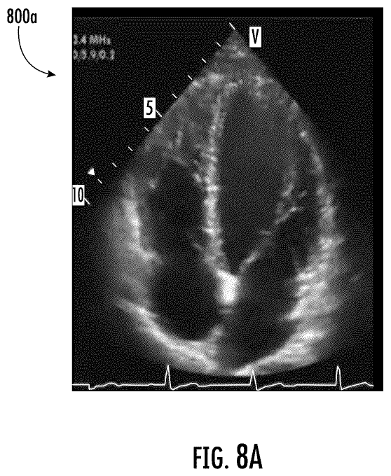

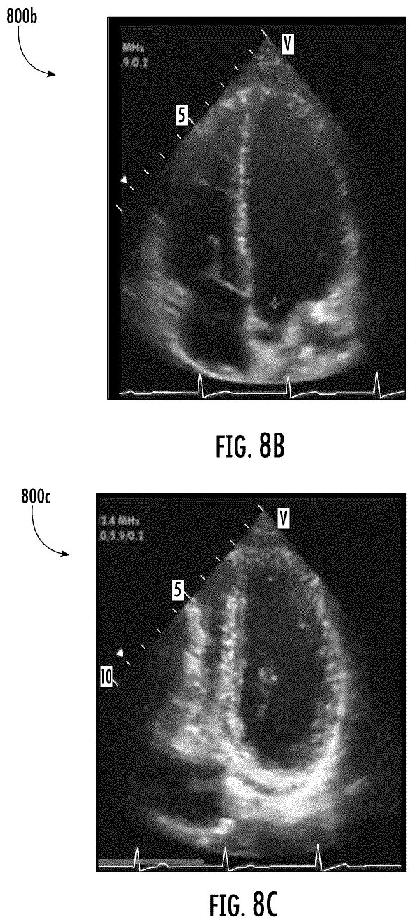

The processor 116 may also be in electronic communication with the display device 132 such that the processor 116 may process ultrasound data obtained by the probe 106 and generate images to display on the display device 132 (e.g., first ultrasound image 800a, second ultrasound image 800b, and third ultrasound image 800c as described below with reference to FIGS. 8A-8C).

As shown in FIG. 1, the processing circuit 114 also includes the memory 118. The memory 118 may be configured to, for example, store processed volumes of data obtained by the ultrasound imaging system 100 (e.g., ultrasound data collected by the probe 106, user inputs received by the user interface 130, etc.). For example, the memory 118 may be a hospital picture archiving and communication system (PACS). The memory 118 (e.g., memory, memory unit, storage device, etc.) may include one or more devices (e.g., RAM, ROM, Flash memory, hard disk storage, etc.) for storing data and/or computer code for completing or facilitating the processes, layers, and modules described in the present application. The memory 118 may be or include tangible, non-transient volatile memory or non-volatile memory. The memory 118 may also include database components, object code components, script components, or any other type of information structure for supporting the activities and information structures described in the present application.

In various embodiments, the memory 118 may have varying capacity (e.g., storage space) across embodiments of the ultrasound imaging system 100. For example, the memory 118 may be configured to store at least 60 minutes' worth of ultrasound data. The ultrasound data may be stored in the memory 118 such that the ultrasound data may be retrieved according to an order/time of acquiring the data. That is, the ultrasound data may be stored with a timestamp indicating a time at which the ultrasound data was collected and may be retrieved starting with an oldest time at which the ultrasound data was collected.

The processing circuit 114 also includes the image processing circuit 120, the AI circuit 122, and the control circuit 124. Both the image processing circuit 120, the AI circuit 122, and the control circuit 124 are configured to facilitate providing recommended imaging parameters during an ultrasound scan, as described herein.

The image processing circuit 120 is configured to receive image data obtained by the transducer of the probe 106 during an ultrasound scan. The image data refers to ultrasound data collected by the probe 106 while performing an ultrasound examination on a patient. For example, the image data collected during an echocardiogram may include images of a patient's heart and specific structures (e.g., ventricles, atria, etc.) therein. The image processing circuit 120 may include multiple deep learning-based models configured to analyze the image data. For example, the image processing circuit may be configured to identify a view from which the image data is captured, an anatomical structure, feature, region, etc. captured by the image data, the presence of a pathology in the image data, and so on. The image processing circuit 120 may be configured to identify the anatomical structure using one or more algorithms (e.g., image processing algorithms such as edge detection, machine learning models, deep neural networks, etc.). In some embodiments, the image processing circuit 120 may identify anatomical features such as bones, blood vessels, organs, etc., based on a shape, relative proximity, apparent depth, orientation, etc. of said features in the image data. Then, based on the identified anatomical features, the image processing circuit 120 may be configured to determine the anatomical structure depicted in the image data. For example, because a lung is not able to be imaged directly (e.g., due to the lung being full of air and the mismatch in the acoustic impedance between the air and the transducer of the probe 106), the image processing circuit 120 may determine that the lung is being imaged based on a movement and/or orientation of the lung relative to surrounding structures, such as the pleura, via a deep learning classification model trained to recognize the movement and/or orientation of the lung, and other anatomical structures. In some embodiments, the image processing circuit 120 may detect movement of structures in the image data by comparing the location, shape, size, etc., of identified structures across a set of images (e.g., a cine loop).

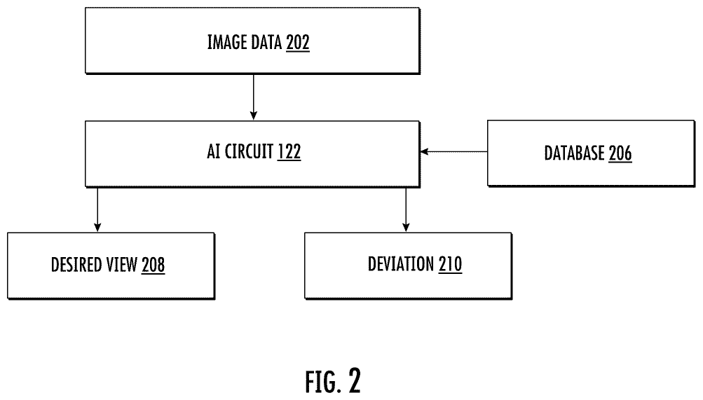

As described in greater detail below, the AI circuit 122 may be configured to adjust or steer the ultrasound signal based on contextual information (e.g., from a database 206, as shown in FIG. 2) regarding a medical imaging procedure. For example, during an ultrasound performed using the ultrasound imaging system 100, the AI circuit 122 may be configured to adjust the ultrasound signal to receive a new image data based on a determination of whether the probe 106 is being navigated to a position in which a diagnostic-quality ultrasound image can be captured or is already positioned in which a diagnostic-quality ultrasound image can be captured.

The ultrasound imaging system 100 may also include an external database 128 and a user interface 130. The external database 128 refers to a database from which the processing circuit 114 (e.g., the AI circuit 122) may retrieve information used to provide recommended imaging parameters during an ultrasound scan. For example, the external database 128 may be a medical information database. The medical information database may store clinical guidelines, standard practices, medical literature, medical textbooks, published research, previous case studies, and so on. Depending on an implementation of the ultrasound imaging system 100 and/or a procedure performed thereby, the AI circuit 122 may retrieve clinical guidelines, standard practices, medical literature, medical textbooks, published research, and previous case studies related to the implementation and/or procedure. For example, if the ultrasound imaging system 100 is being used in a hospital setting to perform an LAA closure procedure, the AI circuit 122 may retrieve clinical guidelines and standard practices related to the hospital setting and the LAA closure procedure. Continuing with this example, the AI circuit 122 may also retrieve information from the medical literature, medical textbooks, published research, and previous case studies related to cardiac anatomy and the LAA closure procedure. In some instances, the information retrieved from the external database 128 may include one or more algorithms used to determine a desired view and determine a deviation from a current view to the desired view.

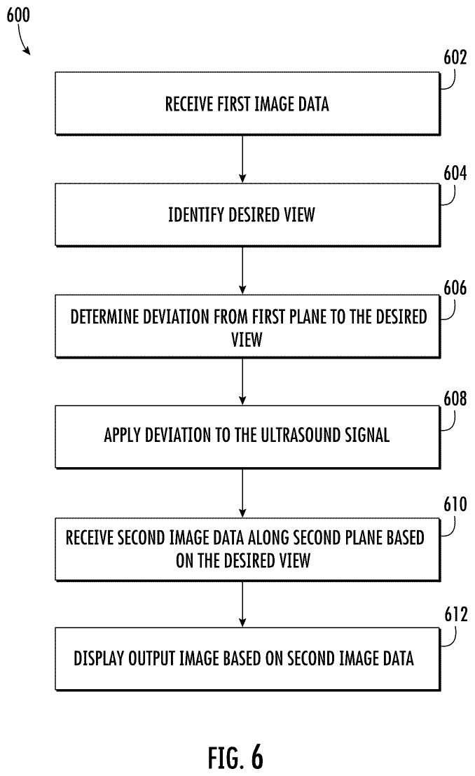

In some embodiments, the image processing circuit 120 and/or the control circuit 124 may be configured to apply one or more algorithms used by the AI circuit 122 and/or retrieved from the external database 128. For example, as described below with reference to FIG. 6, the image processing circuit 120 may use one or more algorithms to assess anatomical features (e.g., using a segmentation model) captured by the image data. In this way, the image processing circuit 120 and the control circuit 124 may be configured to determine a deviation from a plane corresponding to the image data to a desired view based on the analysis of the image data using the AI circuit 122, such that an ultrasound beam can be adjusted using the deviation. The ultrasound beam refers to the transmitted ultrasound signal.

The user interface 130 may be used by a sonographer or other clinician to control operation of the ultrasound imaging system 100. For example, the sonographer may use the user interface 130 to control the input of patient data, to change a scanning or display parameter, to adjust a segmentation of an anatomical feature depicted in an ultrasound image, and/or to select various other modes, operations, parameters, etc. of the ultrasound imaging system 100. In some embodiments, the user interface 130 may include an off-the-shelf consumer electronic device such as a smartphone, a tablet, a laptop, and so on. For the purposes of this disclosure, the term “off-the-shelf consumer electronic device” is defined to be an electronic device that was designed and developed for general consumer use and one that was not specifically designed for use in a medical environment. Alternatively, in other embodiments, the user interface 130 may be an electronic device that was designed and developed for use in a medical environment.

According to some embodiments, the user interface 130 may be physically separate from the rest of the ultrasound imaging system 100 (e.g., the transmit beamformer 102, the transmitter 104, the probe 106, the receiver 110, the receive beamformer 112, the processing circuit 114, and/or the external database 128). The user interface 130 may communicate with the processor 116 through a wireless protocol, such as Wi-Fi, Bluetooth, wireless local area network (WLAN), near-field communication, and so on. According to some embodiments, the user interface 130 may communicate with the processor 116 through an application programming interface (API).

In some embodiments, the user interface 130 may include physical controls such as one or more of buttons, sliders, a rotary knob, a mouse, a keyboard, a trackball, hard keys linked to specific actions, soft keys that may be configured to control different functions, and so on. As shown in FIG. 1, the user interface 130 may also include a display device 132. In some embodiments, the display device 132 may be configured to display a graphical user interface (GUI) based on an instruction from the memory 118. The GUI may include user interface icons representing commands and instructions relating to the operation of the ultrasound imaging system 100. The user interface icons of the GUI may be configured such that a user (e.g., the sonographer, clinician, etc.) may select a specific user interface icon in order to initiate a specific function controlled by the GUI. For example, various user interface icons may be used to represent windows, menus, buttons, cursors, scroll bars, and so on. That is, the physical controls of the user interface 130 may be included as individual hardware elements, as user interface icons displayed on the display device 132, or as a combination of hardware elements and user interface icons.

In some embodiments, the display device 132 may include a touch-sensitive display device or a touch screen. According to such embodiments, the touch screen may be configured to interact with the GUI displayed by the display device 132 such that a user (e.g., the sonographer) can interact with the GUI via the touch screen. The touch screen may be a single-point touch screen that is configured to detect a single contact point at a time, or the touch screen may be a multi-point touch screen that is configured to detect multiple points of contact at a time. For embodiments where the touch screen is a multi-point touch screen, the touch screen may be configured to detect multi-point gestures involving contact from two or more of a user's fingers at a time. The touch screen may be a resistive touch screen, a capacitive touch screen, or any other type of touch screen that is configured to receive inputs from a stylus or one or more of a user's fingers. According to some embodiments, the touch screen may be an optical touch screen that uses technology such as infrared light or other frequencies of light to detect one or more points of contact initiated by a user. In some embodiments, the touch screen may be incorporated as part of the display device 132 or may be separate from the display device 132. The user interface 130 may also include a proximity sensor configured to detect objects and/or gestures that are within a predetermined distance (e.g., five feet, six inches, ten centimeters, etc.) of the proximity sensor. In various embodiments, the proximity sensor may be located on the display device 132 or as part of a touch screen that is separate from the display device 132.

Referring to FIG. 2, the AI circuit 122 of the ultrasound imaging system 100 is shown in greater detail. The AI circuit 122 is configured to determine a desired view 208 based on image data 202, as described with reference to step 604 of method 600. The AI circuit 122 can be trained to recognize a closest standard view, the desired view 208, corresponding to an acquired image data along a plane. For example, the AI circuit 122 may determine the desired view 208 through the use of an image library including a plurality of historical ultrasound images. As described herein, the image library may be used to train the AI circuit 122. For example, the image library may aid the AI circuit 122 in processing the image data 202 to determine a view of the image data 202 and/or determining the desired view 208.

The AI circuit 122 is further configured to determine a deviation 210 from a plane corresponding to image data 202 to a desired view. As described with reference to step 606 of method 600, the deviation indicates a difference of the plane to the desired view in terms of a linear movement, rotation, or tilt. It should be understood that the deviation can be expressed as any combination of a value, an angle, a direction, etc. In some embodiments, the deviation is determined by comparing the image data 202 and/or the plane to an image library including a plurality of historical ultrasound images. In some embodiments, the deviation is determined by a machine learning model.

The AI circuit 122 may provide feedback that directs a user (e.g., a sonographer) to obtain the desired view or close enough to the desired view in order to automatically apply a deviation to reach the desired view. In some embodiments, the feedback is based on clinic guidelines for obtaining ultrasound images for determining a particular diagnosis. Thus, because the image data 202 received to determine the deviation may not be in the desired view and the needed output from the user, a reduced-quality image can be generated or displayed of the image data 202 as the user receives the image data 202 and navigates the probe according to the feedback generated by the AI circuit 122. For example, while the probe 106 is being navigated into a position to apply the deviation to an ultrasound signal according to feedback generated by the AI circuit 122, the user may not be relying on the displayed ultrasound image to determine whether an acceptable view is being achieved. As such, in order to reduce battery consumption of the probe 106, thereby extending a battery life of the probe 106, the ultrasound image displayed on the display device 132 while the probe 106 is being navigated may be reduced in quality via the AI circuit 122.

As shown, the AI circuit 122 receives information from a database 206 regarding ultrasound imaging. The database 206 can provide contextual information for the AI circuit 122. For example, the database 206 can include an image library including a plurality of historical ultrasound images. For example, the ultrasound images may include standard views of anatomies and non-standard views of anatomies. Additionally, the database 206 may include a plurality of deviation values and example application of deviations onto planes. In some embodiments, the database 206 may include an indication of whether the probe 106 is currently positioned such that a desired view of the anatomy can be captured or whether the probe 106 is currently being navigated (e.g., by the user or automatically) to obtain an image in the desired view. In other embodiments, as described with reference to method 600, the database 206 may include contextual information for additional image data taken from a same scan volume as the image data 202 in order to provide additional information to determine the deviation. In various embodiments, the database 206 is a part of the external database 128.

The database 206 may be used as an input to the AI circuit 122. In some embodiments, the AI circuit 122 may include a Bayesian neural network. As shown in FIG. 2, the AI circuit 122 is configured to generate the desired view 208 and the deviation 210 based on the received information from the database 206. In some embodiments, the AI circuit 122 may be trained using information regarding historic ultrasound scans performed by expert sonographers (e.g., sonographers with specific qualifications, sonographers having a number of years of experience, etc.). For example, an expert sonographer may conduct an echocardiogram or other ultrasound (e.g., a fetal ultrasound, etc.). Information regarding the echocardiogram (e.g., the imaging parameters used while the probe 106 is being navigated to the desired view, a position of the anatomical feature in the desired view, an AI score for the images during image capture in the desired view, etc.) may be stored (e.g., in the memory 118) in the database 206 as contextual information.

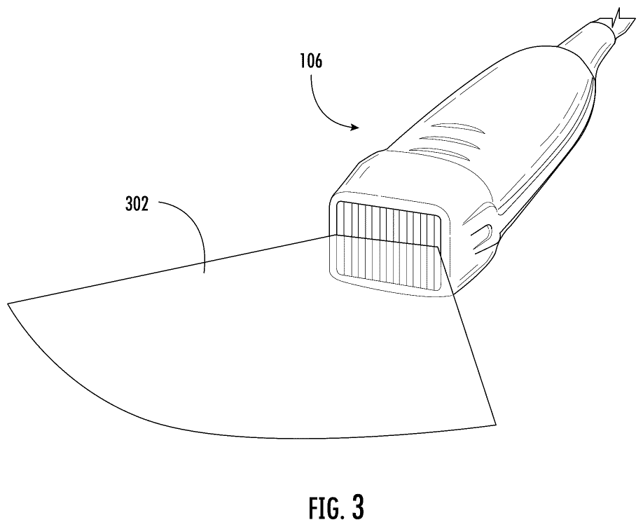

Referring to FIG. 3, an illustration of an ultrasound imaging probe is shown, according to an example embodiment. The ultrasound imaging probe 106 is configured for two-dimensional ultrasound imaging. As such, the ultrasound imaging probe 106 can transmit and receive an ultrasound signal to provide image date along a scan plane 302. The scan plane 302 is limited and a user can adjust the scan plane 302 by physically rotating, tilting, or translating a position of the ultrasound imaging probe.

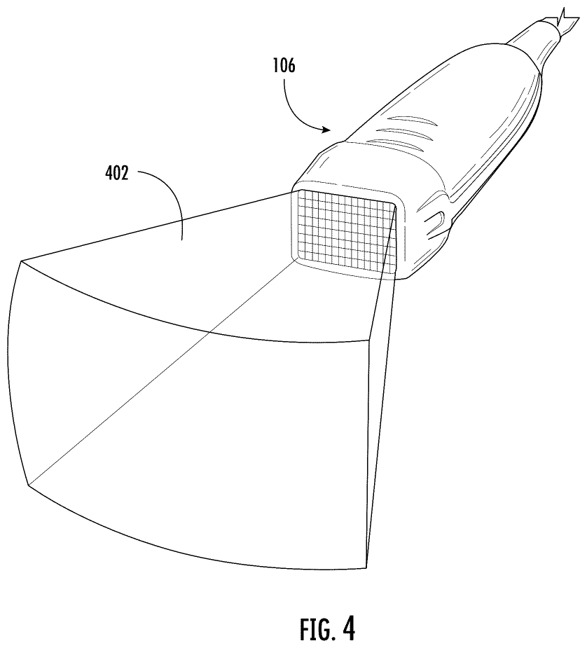

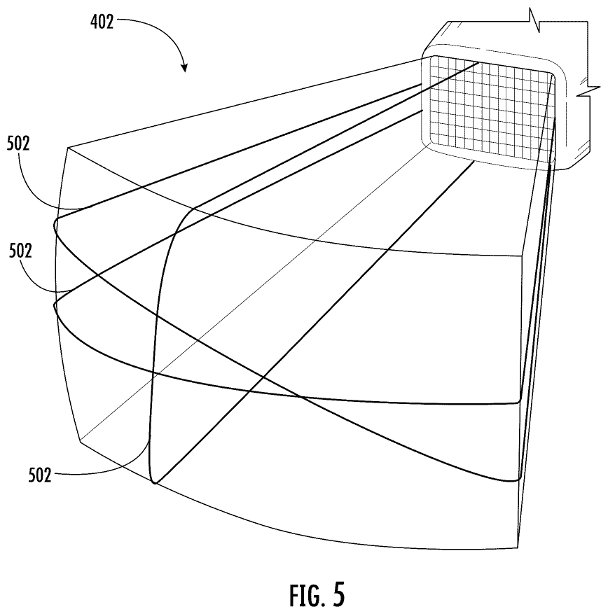

Referring to FIG. 4, an illustration of an ultrasound imaging probe is shown, according to an example embodiment. The ultrasound imaging probe 106 is configured for two-dimensional ultrasound imaging and three-dimensional ultrasound imaging. In contrast to the probe 106 of FIG. 3, the probe 106 is capable of transmitting and receiving an ultrasound signal in a scan volume 402. In comparison to the scan plane 302, the scan volume 402 allows for electronic steering of the ultrasound signal in order to receive multiple scan planes. For example, the ultrasound signal can be titled, rotated, or translated in order to receive a scan plane (e.g., scan planes 502 as shown in FIG. 5) within the scan volume 402.

Referring to FIG. 5, an illustration of the scan volume 402 of the ultrasound imaging probe of FIG. 4 is shown. As depicted in the figure, the scan volume 402 can be used to retrieve image data along a number of scan planes 502. A scan plane within the scan volume 402 can be automatically steered electronically due to receiving new image data from a new location within the scan volume 402. Due to this, as described herein, an addition of artificial intelligence can aid in determining how the scan plane should be steered to receive image data that is preferred or along a standard view. For a probe configured for only two-dimensional imaging, the scan plane cannot be electronically steered, and a user would have to manually adjust the probe to receive image data, which can be a tedious and unsuccessful process. With the combination of a three-dimensional probe and artificial intelligence, a scan plane can be automatically manipulated to achieve a desired view.

Referring to FIG. 6, a flow chart illustrating a method 600 for ultrasound imaging using an ultrasound imaging system is shown. In at least one embodiment, the ultrasound imaging system referred to by method 600 is the ultrasound imaging system 100 described above with reference to FIG. 1, and method 600 may be implemented by the ultrasound imaging system 100. In some embodiments, method 600 may be implemented as executable instructions in a memory of the ultrasound imaging system 100, such as the memory 118 of FIG. 1.

As shown in FIG. 6, at step 602, method 600 includes receiving first image data. As described herein, the transducer of the ultrasound imaging system 100 is configured to transmit and receive an ultrasound signal in a three dimensional volume. The three-dimensional volume includes a plurality of planes in a number of views (e.g., a transverse view, a coronal view, a sagittal view). The first image data may be received from extracting slices from the volume. Processing of the ultrasound signals allows for image construction. Processing ultrasound signals may include converting the received echoes from the probe 106 into electrical signals, passing the electrical signals through the receive beamformer 112 for ultrasound data, and performing beamforming techniques to generate an image. At step 602, the first image data is obtained by a probe (e.g., probe 106, a three-dimensional probe) from the three-dimensional volume. The first image data corresponds with a first plane in the volume. For example, the first plane may be along a transverse view. In various embodiments, the image processing circuit 120 applies processing techniques to the constructed image to further improve quality of the constructed images (e.g., contrast adjustment, brightness adjustment, noise reduction, etc.).

As shown in FIG. 6, method 600 includes identifying a desired view based on the first image data at step 604. The desired view may be a standard view for the type of imaging done.

For example, for a lung ultrasound, the desired view for scanning a pleura may be a view in which the pleura is perpendicular to the ultrasound signal. The desired view may be a standard view known in practice. Identification of the desired view provides contextual information to an artificial intelligence model (e.g., the AI circuit 122) on what is desired such that the artificial intelligence model can aid in determining how to get to the desired view.

In various embodiments, the method 600 includes identifying an anatomical feature based on the first image data. In some embodiments, this may be performed by the processing circuit 114. For example, the first image data may be obtained during a lung ultrasound, and therefore the first anatomical feature may include a pleura of the patient being imaged. It should be appreciated, however, that although the ultrasound imaging system 100 cannot capture an image of the lung cavity due to the lung cavity being filled with air, an image of the lung cavity may be derived using surrounding anatomical structures/features (e.g., ribs, the pleura, etc.) depicted in the image data. Identifying the anatomical feature may include segmentation, analysis, and mathematical models in order to properly identify the feature. In various embodiments, the desired view is based on a position of the anatomical feature. For example, the position of the anatomical feature in the first image data may aid in determining a desired position of the anatomical feature, allowing for the desired view to be determined. In various embodiments, the anatomical feature may aid in determining the desired view. Continuing the previous example with the lung ultrasound, identifying the anatomical feature as the pleura may aid in determining the desired view.

In various embodiments, artificial intelligence is used for identifying the desired view. For example, identifying the desired view may be based on an output of an artificial intelligence model (e.g., the AI circuit 122). The artificial intelligence model may be trained to recognize standard views corresponding with image data. The artificial intelligence model may then generate the output corresponding to the desired view using the first image data. The artificial intelligence model may use data from the first image data (e.g., position data, anatomical features etc.) to generate the output.

In various embodiments, a patient's history and/or data may be used to identify the desired view. For example, the desired view may be a view of the patient's previous scan. This may be beneficial for a variety of applications, such as imaging of the lung. When treating a patient, it is important to properly visualize B-lines and the pleura to monitor a state of the patient. By capturing the same view for each scan, such that the desired view is the view of the patient's previous scan, consistent tracking of the patient can be ensured. In various embodiments, the artificial intelligence model may be used to determine a view of the patient's previous scan.

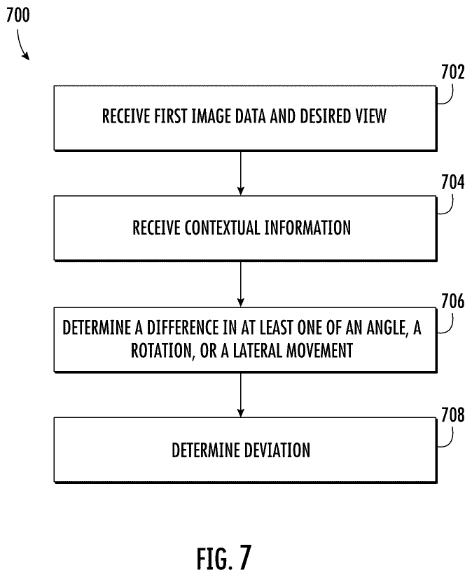

As shown in FIG. 6, method 600 includes determining a deviation from the first plane to the desired view based on the first image data at step 604. Step 604 utilizes the artificial intelligence model (e.g., AI circuit 122). The deviation includes information on how the first plane should be adjusted to reach the desired view. Determining the deviation may include determining a difference in at least one of an angle (e.g., tilt), a rotation, or a lateral movement between the first plane and the desired view, such that the deviation is a combination of the difference. For example, as describe with reference to FIG. 7, the artificial intelligence model may determine, based on the database 206, that the first plane differs from the desired view by a tilt of a first degree and a rotation of a second degree such that the first plane must be tilted by the first degree and rotated by the second degree to reach the desired view.

In various embodiments, the artificial intelligence model may use additional information from the three-dimensional volume to determine the deviation. For example, the method may further include receiving third image data from the three-dimensional volume. The third image data is obtained from a third plane at an angle relative to the first plane. For example, the angle of the third plane relative to the first plane can be 90°. In various embodiments, additional image data (e.g., a fourth image data, a fifth image data, a plurality of image data, etc.) is received from the volume. The additional image data is obtained from an additional plane (e.g., a fourth plane, a fifth plane, a plurality of planes, etc.) at a second angle relative to the first plane. For example, the angle of the third plane relative to the first plane and the second angle of a fourth plane relative to the first plane can be 60°. The third image data and additional image data may provide the artificial intelligence model more information and context to determine the deviation. For example, the third plane may be compared with an additional plane that is relative to the desired view by the angle to aid in determining the deviation. As the third image data does not have to be displayed, in various embodiments, an image rate of the third image data does not have to optimal for viewing. For example, the first image data can be received at a first image rate and the third image data can be received at a second image rate. In various embodiments, the second image rate is lower than the first image rate.

As shown in FIG. 6, method 600 includes automatically applying the deviation to the ultrasound signal at step 608. In various embodiments, the processing circuit 114 may be used to apply to the deviation to the ultrasound signal. Automatically applying the deviation can include automatically steering the ultrasound signal (e.g., beam steering). For example, electronic rotation and tilting can be implemented to apply the deviation. Automatic steering of the ultrasound signal can include modifying the ultrasound signal to modify at least one of a rotation or tilt of an imaging plane. Additionally, the transmit beamformer 102 may be automatically adjusted to adjust the ultrasound beam, therefore modifying the ultrasound signal. As described herein, the usage of three-dimensional imaging produces a scan volume in which steering of the transmitted ultrasound signal (e.g., the ultrasound beam) is possible to reach a number of planes.

Applying the deviation to the ultrasound signal allows for second image data to be received at step 610. The second image data corresponds with a second plane based on the desired view, such that the second image data is the standard view, the correctly aligned view, etc. In other words, a produced image from the second image data may result in an improvement in analysis of ultrasound images, including measurements and diagnosis. In various embodiments, the second image data is received at an image rate that is greater than the first image rate.

As shown in FIG. 6, method 600 includes displaying an output image based on the second image data at step 612. In some embodiments, the output image is displayed on the display device 132. In various embodiments, the output image includes an indication of the deviation. For example, an operator of the ultrasound imaging system 100 can view the indication to view how the first image data and/or the ultrasound signal was adjusted to receive the second image data. Beneficially, this can provide the operator with additional information on the first image data and second image data.

In various embodiments, the deviation may be greater than what can be automatically done by the ultrasound imaging system 100. For example, the artificial intelligence model may determine that the deviation requires the operator to adjust the probe before the deviation can be automatically applied to the ultrasound signal. As such, the method may include providing feedback to a user (e.g., the operator) to manually adjust a position of the probe based on the deviation. In various embodiment, the feedback is provided on the display device 132. For example, the operator could be prompted by the display device 132 to tilt the probe 106 by X degrees along an axis in a first direction or by Y degrees along the axis in a second direction opposite of the first direction such that the tilting of the probe 106 results in placing the probe at the position to apply the deviation. Other movements of the probe 106, including rotating, sliding, rocking, etc., may be provided to the operator. In some embodiments, the user interface 130 could prompt the operator to adjust the probe 106 by providing an audio output. In various embodiments, the user interface 130 may provide real-time feedback as the operator adjust the probe.

In various embodiments, upon receiving the second image data, the processing circuit 114 and/or the artificial intelligence model may determine that the second image data differs significantly from a standard view. For example, the standard view may be a new standard view may be determined by the artificial intelligence model or the standard view may be the desired view. As such, the method 600 may be iterative and using the second image data and the standard view, the method 600 can determine a deviation from the second plane of the second image data to the standard view, as described with reference to step 606. This deviation can then be applied to the ultrasound signal (e.g., step 608) to receive a third image data along a third plane based on the standard view (e.g., step 610).

Referring to FIG. 7, a method 700 for determining a deviation from the first plane to the desired view is shown. The method 700 utilizes the artificial intelligence model (e.g., the AI circuit 122) and the database 206 to determine the deviation.

As shown in FIG. 7, method 700 includes receiving first image data and desired view at step 702. The first image data and the desired view are received by the artificial intelligence model. The first image data and the desired view, as described with reference to FIG. 2 and method 600, provide information to the artificial intelligence model that is used to determine the deviation.

As shown in FIG. 7, method 700 includes receiving, by the artificial intelligence model, contextual information at step 704. As described herein, contextual information can be provided to gain more information on the image data and the desired view in order to determine the deviation. The contextual information can include an image library including a plurality of historical ultrasound images. For example, the ultrasound images may include standard views of anatomies and non-standard views of anatomies. The contextual information is important for understanding a state of the patient and determining differences or anything unusual of the image data as the image data may be compared with historical data. In various embodiments, the contextual information includes patient information.

As shown in FIG. 7, method 700 includes determining a difference in at least one of angle, a rotation, or a lateral movement at step 706. As described herein with reference to step 606 of method 600, the deviation includes information on how the first plane should be adjusted to reach the desired view. The artificial intelligence model may determine, based on the database 206 and the first image data, that the first plane differs from the desired view by an angle, a rotation, and/or a lateral movement. In various embodiments the difference is determined using position information of the desired view and the image data. In various embodiments, calculations to determine the difference along the direction of the movements from the first plane to the desired view may be determined. For example, the first plane may differ from the desired view such that the first plane must be moved laterally and rotated 10°.

As shown in FIG. 7, method 700 includes determining the deviation at step 708. The deviation may be a combination of the differences. Continuing the example discussed with reference to step 706, the deviation of the first plane may be lateral movement by X and a rotation of 10°.

Referring to FIGS. 8A-8C, a three-dimensional ultrasound image of a first ultrasound image 800a, a second ultrasound image 800b, and a third ultrasound image 800c is show. The ultrasound images are extracted from a three-dimensional volume obtained from transmitting and receiving an ultrasound signal. The ultrasound images depict a cardiac ultrasound. For cardiac ultrasound, it is important for the operator to find a standard view and orientation by translation, tilting, and rotating. Finding the standard view, as described herein, is important for having accurate measurements and correct diagnosis. FIG. 8A shows the first ultrasound image 800a with an aligned Apical 4 chamber view of a heart. The second ultrasound image 800b and the third ultrasound image 800c show incorrectly aligned Apical 4 chamber views (e.g., the probe has been tilted such that acquisition is off-axis to the aligned view, or correctly positioning a 2D scan plane in 3D space). Using the method 600, as described herein, the tilt and rotation to adjust the second ultrasound image 800b and the third ultrasound image 800c to the correct view of the first ultrasound image 800a can be done with the artificial intelligence model and a three-dimensional ultrasound probe.

The embodiments described herein have been described with reference to drawings. The drawings illustrate certain details of specific embodiments that provide the systems, methods and programs described herein. However, describing the embodiments with drawings should not be construed as imposing on the disclosure any limitations that may be present in the drawings.

It should be understood that no claim element herein is to be construed under the provisions of 35 U.S.C. § 112(f), unless the element is expressly recited using the phrase “means for.”

As utilized herein, terms of degree such as “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to any precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that terms such as “exemplary,” “example,” and similar terms, as used herein to describe various embodiments, are intended to indicate that such embodiments are possible examples, representations, or illustrations of possible embodiments, and such terms are not intended to connote that such embodiments are necessarily extraordinary or superlative examples.

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any element on its own or any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the drawings. It should be noted that the orientation of various elements may differ according to other exemplary embodiments, and that such variations are intended to be encompassed by the present disclosure.

As used herein, terms such as “engine” or “circuit” may include hardware and machine-readable media storing instructions thereon for configuring the hardware to execute the functions described herein. The engine or circuit may be embodied as one or more circuitry components including, but not limited to, processing circuitry, network interfaces, peripheral devices, input devices, output devices, sensors, etc. In some embodiments, the engine or circuit may take the form of one or more analog circuits, electronic circuits (e.g., integrated circuits (IC), discrete circuits, system on a chip (SOCs) circuits, etc.), telecommunication circuits, hybrid circuits, and any other type of circuit. In this regard, the engine or circuit may include any type of component for accomplishing or facilitating achievement of the operations described herein. For example, an engine or circuit as described herein may include one or more transistors, logic gates (e.g., NAND, AND, NOR, OR, XOR, NOT, XNOR, etc.), resistors, multiplexers, registers, capacitors, inductors, diodes, wiring, and so on).

An engine or circuit may be embodied as one or more processing circuits comprising one or more processors communicatively coupled to one or more memory or memory devices. In this regard, the one or more processors may execute instructions stored in the memory or may execute instructions otherwise accessible to the one or more processors. The one or more processors may be constructed in a manner sufficient to perform at least the operations described herein. In some embodiments, the one or more processors may be shared by multiple engines or circuits (e.g., engine A and engine B, or circuit A and circuit B, may comprise or otherwise share the same processor which, in some example embodiments, may execute instructions stored, or otherwise accessed, via different areas of memory).

Alternatively or additionally, the one or more processors may be structured to perform or otherwise execute certain operations independent of one or more co-processors. In other example embodiments, two or more processors may be coupled via a bus to enable independent, parallel, pipelined, or multi-threaded instruction execution. Each processor may be provided as one or more suitable processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor, etc.), microprocessor, etc. In some embodiments, the one or more processors may be external to the apparatus, for example the one or more processors may be a remote processor (e.g., a cloud based processor). Alternatively or additionally, the one or more processors may be internal and/or local to the apparatus. In this regard, a given engine or circuit or components thereof may be disposed locally (e.g., as part of a local server, a local computing system, etc.) or remotely (e.g., as part of a remote server such as a cloud based server). To that end, engines or circuits as described herein may include components that are distributed across one or more locations.

An example system for providing the overall system or portions of the embodiments described herein might include one or more computers, including a processing unit, a system memory, and a system bus that couples various system components including the system memory to the processing unit. Each memory device may include non-transient volatile storage media, non-volatile storage media, non-transitory storage media (e.g., one or more volatile and/or non-volatile memories), etc. In some embodiments, the non-volatile media may take the form of ROM, flash memory (e.g., flash memory such as NAND, 3D NAND, NOR, 3D NOR, etc.), EEPROM, MRAM, magnetic storage, hard discs, optical discs, etc. In other embodiments, the volatile storage media may take the form of RAM, TRAM, ZRAM, etc. Combinations of the above are also included within the scope of machine-readable media. In this regard, machine-executable instructions comprise, for example, instructions and data which cause a general purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components, etc.), in accordance with the example embodiments described herein.

Although the drawings may show and the description may describe a specific order and composition of method steps, the order of such steps may differ from what is depicted and described. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the appended claims. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes and omissions may be made in the design, operating conditions, and arrangement of the embodiments without departing from the scope of the present disclosure as expressed in the appended claims.

1. An ultrasound imaging system comprising:
a transducer configured to transmit and receive an ultrasound signal in a three-dimensional volume, the three-dimensional volume comprising a plurality of planes; and
a processing circuit having a processor coupled to a memory device storing instructions thereon that, when executed, cause the processing circuit to perform operations comprising:
receiving first image data obtained by an ultrasound probe from the three-dimensional volume, the first image data corresponding with a first plane in the volume;
identifying a desired view based on the first image data;
determining, by an artificial intelligence model, a deviation from the first plane to the desired view based on the first image data;
automatically applying the deviation to the ultrasound signal to receive second image data corresponding with a second plane based on the desired view; and
displaying an output image based on the second image data on a display screen of the ultrasound imaging system.

2. The ultrasound imaging system of claim 1, wherein identifying the desired view is based on an output from the artificial intelligence model.

3. The ultrasound imaging system of claim 1, wherein the ultrasound probe is a three-dimensional ultrasound matrix probe.

4. The ultrasound imaging system of claim 1, wherein automatically applying the deviation to the ultrasound signal comprises automatically steering the ultrasound signal.

5. The ultrasound imaging system of claim 4, wherein automatically steering the ultrasound signal comprises modifying the ultrasound signal to modify at least one of a rotation or tilt of an imaging plane.

6. The ultrasound imaging system of claim 1, wherein determining the deviation comprises determining a difference in at least one of an angle, a rotation, or a lateral movement between the first plane and the desired view.

7. The ultrasound imaging system of claim 1, wherein the operations further comprise receiving third image data obtained by the ultrasound probe from the three-dimensional volume, the third image data obtained from a third plane at an angle relative to the first plane, the deviation from the first plane to the desired view determined based on the first image data and the third image data.

8. The ultrasound imaging system of claim 7, wherein the angle of the third plane relative to the first plane is 90°.

9. The ultrasound imaging system of claim 7, wherein the first image data is received at a first image rate and the third image data is received at a second image rate lower than the first image rate.

10. The ultrasound imaging system of claim 1, wherein the output image comprises an indication of the deviation.

11. The ultrasound imaging system of claim 1, wherein the operations further comprise identifying an anatomical feature based on the first image data, the desired view based on a position of the anatomical feature.

12. The ultrasound imaging system of claim 1, wherein the operations further comprise providing feedback to a user to manually adjust a position of the ultrasound probe based on the deviation.

13. A medical imaging system, comprising:
an ultrasound probe configured to transmit and receive an ultrasound signal in a three-dimensional volume to obtain first image data along a first plane; and
a control circuit configured to determine, by an artificial intelligence model, a deviation from the first plane to a desired view based on the first image data;
automatically applying the deviation to the ultrasound signal to receive second image data corresponding with a second plane based on the desired view.

14. The medical imaging system of claim 13, wherein identifying the desired view is based on an output from the artificial intelligence model.

15. The medical imaging system of claim 13, wherein the medical imaging system comprises an image processing circuit configured to identify an anatomical feature based on the first image data, the desired view based on a position of the anatomical feature.

16. The medical imaging system of claim 13, wherein automatically applying the deviation to the ultrasound signal comprises automatically steering the ultrasound signal.

17. The medical imaging system of claim 13, wherein the ultrasound probe is configured to receive third image data from the three-dimensional volume, the third image data obtained from a third plane at an angle relative to the first image data, the deviation from the first plane to the desired view determined based on the first image data and the third image data.

18. A method comprising:
receiving, by a processing circuit, first image data obtained by an ultrasound probe from a three-dimensional volume, the first image data corresponding with a first plane in the volume;
identifying, by the processing circuit, a desired view based on the first image data;
determining, by the processing circuit and an artificial intelligence model, a deviation from the first plane to the desired view based on the first image data;
automatically applying, by the processing circuit, the deviation to an ultrasound signal to receive second image data corresponding with a second plane along the desired view; and
displaying an output image based on the second image data on a display screen of an ultrasound imaging system.

19. The method of claim 18, wherein automatically applying the deviation to the ultrasound signal comprises automatically steering the ultrasound signal by modifying at least one of a rotation or a tilt of an imaging plane of the ultrasound signal.

20. The method of claim 18, further comprising receiving third image data obtained by the ultrasound probe from the three-dimensional volume, the third image data obtained from a third plane at an angle relative to the first plane, the deviation from the first plane to the desired view determined based on the first image data and the third image data.
